(19) United States
(12) Patent Application Publication
(10) Pub. No.: US 2004/0242864 A1
(43) Pub. Date: Dec. 2, 2004

(54) Process for the preparation of crystalline cefuroxime axetil

(76) Inventors: Davide Longoni, Gorgonzola (IT); Marco Alpegiani, Milano (IT); Walter Cabri, Rozzano (IT); Claudio Felisi, Zelo Buon Persico (IT)

Correspondence Address: ROTHWELL, FIGG, ERNST & MANBECK, P.C., 1425 K STREET, N.W., SUITE 800, WASHINGTON, DC, 20005, US

(21) Appl. No.: 10/489,321

(22) PCT Filed: Sep. 4, 2002
(86) PCT No.: PCT/EP02/09896

(30) Foreign Application Priority Data
Sep. 14, 2001 (IT) MI2001A001925

(51) Int. Cl. C07D 501/14

(52) U.S. Cl. 540/222

(57) ABSTRACT

The present invention relates to a process for the preparation of crystalline cefuroxime axetil with high purity lecel and optimal diastereomeric ratio. Said process, which comprises the use of a dimethyl carbonate for isolating crystallizing cefuroxime axetil, is particularly suitable for implementing on an industrial scale.

The present invention relates to a process for the preparation of crystalline cefuroxime with high purity level and optimal diastereomeric ratio.

The process according to the invention yields cefuroxime axetil in high yields and advantageously in terms of costs and safety on an industrial scale.

Cefuroxime axetil, whose non-proprietary name is (R,S)-1-acetoxyethyl (Z)-3-carbamoyloxymethyl-7-[2-(2-furyl)-2-(methoxyimino)-acetamido]-8-oxo-5-thia-1-azabicyclo[4.2.0]oct-2-ene-2-carboxylate, (Formula I) is the 1-acetoxyethyl ester of cefuroxime, a second-generation semisynthetic cephalosporin characterized by a broad spectrum activity against Gram-positive and Gram-negative bacteria. It is orally active and is marketed in the amorphous form, having this physical state better pharmacokinetic/pharmacodynamic characteristics than the crystalline product.

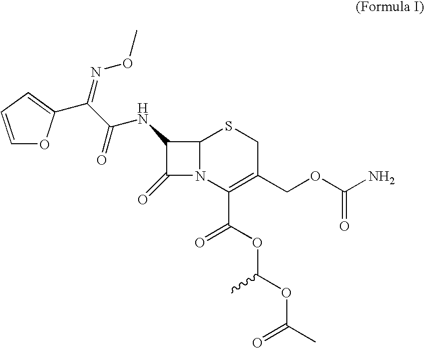

The conventional process for the preparation of cefuroxime axetil is the esterification of cefuroxime with 1-acetoxyethyl bromide (1-bromoethyl acetate), as disclosed in U.S. Pat. No. 4,267,320, to afford, in normal conditions, a crystalline product. The latter is transformed into the amorphous form using special techniques, as described, for example in U.S. Pat. Nos. 4,562,181; 4,820,833; 4,994,467 and 5,103,833. In the processes for the conversion of the crystalline product into the amorphous one, such as spray drying, freeze drying, roller drying or treatment with excipients, the chemical quality of the amorphous product in terms of impurities and diastereomeric ratio is directly related to that of the crystalline precursor, as such processes do not comprise further purification steps.

The commercial product consists of a mixture of two diastereoisomers which should be present in a well-defined ratio: the ratio of A isomer to the sum of the A+B isomers should range from 0.48 and 0.55 [A/(A+B)=0.48÷0.55], as reported in European and United States Pharmacopoeias.

Furthermore, known impurities, such as delta-2 and anti isomers, as well as any unknown impurities should not be present or at least be present in very small amounts and anyway within the limits established by the various Pharmacopoeias.

The process of the present invention allows to obtain cefuroxime axetil with high yields, optimal diastereomeric ratio and high purity level, and can easily be implemented on an industrial scale; the process is in fact carried out in conditions which agree with large-scale operations, using easily available solvents involving no risks for either the handlers or the environment.

The process of the invention comprises:

reacting cefuroxime sodium with 1-acetoxyethyl bromide in a polar aprotic solvent;

partitioning the reaction mixture between water and dimethyl carbonate (DMC);

separating the organic phase, which can optionally be washed with water, decolourized and concentrated;

precipitating the product by treatment with an alkane.

The reaction of cefuroxime sodium with 1-acetoxyethyl bromide is carried out in polar aprotic solvents, such as N-methylpyrrolidone, N,N-dimethylacetamide, N,N-dimethylformamide, dimethylsulfoxide, at a temperature ranging from −5° C. to +30° C. for a time ranging from 30 minutes to 24 h. Completion of the reaction may be assisted by addition of an alkali or alkaline-earth metal carbonate or bicarbonate.

Quenching is carried out by partitioning the reaction mixture between water and DMC at a temperature ranging from +5 to +30° C. After separation of the phases, the organic layer can be washed once or more times with water or with sodium chloride or sodium bicarbonate aqueous solutions or with a sodium phosphate buffer solution. The combined aqueous phases can be extracted with DMC.

If necessary, a further treatment of the organic phase with active charcoal or decolourizing resin may be carried out for a time ranging from 10 minutes to 1 hour, subsequently filtering off the decolourizing agent. The organic phase can be concentrated under vacuum. The final volume of the organic phase, compared with the weight of cefuroxime sodium used in the reaction, can range from 2:1 to 10:1.

Precipitation of cefuroxime axetil partly occurs during concentration of the organic phase and is completed by addition of an alkane or cycloalkane as antisolvent, selected from n-hexane, cyclohexane, n-heptane, methylcyclohexane, n-octane, isooctane. This addition is carried out gradually, under stirring, for a time usually ranging from 30 minutes to 5 h and at a temperature ranging from 10° C. to 40° C. The optimal ratio of antisolvent to concentrated organic phase, to obtain the intended diastereomeric percentage (R,S) and yield in cefuroxime axetil, usually ranges from 1:5 to 10:1 volume/volume.

The following example illustrates the invention in detail.

### EXAMPLE

110.8 kg of cefuroxime sodium and 612 l of N,N-dimethylacetamide are placed in a suitable reactor, under nitrogen atmosphere. The mixture is cooled to 0÷5° C., and added with 77 kg of 1-bromoethyl acetate, then with 0.027 kg of potassium carbonate under vigorous stirring. The reaction is completed in some hours. Afterwards, a mixture consisting of 1600-1700 l of DMC and 1110 l of a 3% NaHCO₃ aqueous solution is added thereto, then the phases are separated. The organic phase is washed to neutral pH. The solution is concentrated to one third of the volume at T<25° and 550 l of n-hexane are added. After completion of the crystallization, the product is filtered and dried at 50° C. under vacuum to constant weight to obtain 105 kg of dry product (diastereomeric ratio: 0.52; HPLC purity≈99.6%).

Weight yield=95.0%

1. A process for the preparation of crystalline cefuroxime axetil, which comprises:
reacting cefuroxime sodium with 1-acetoxyethyl bromide in a polar aprotic solvent;
partitioning the reaction mixture between water and dimethyl carbonate (DMC);
separating the organic phase, which can optionally be washed with water, decolourized and concentrated;
precipitating the product by treatment with an alkane.

2. A process as claimed in claim 1, wherein the polar aprotic solvent is selected from N-methylpyrrolidone, N,N-dimethylacetamide, N,N-dimethylformamide and dimethylsulfoxide.

3. A process as claimed in claim 1, wherein the alkane used for the precipitation is selected from n-hexane, cyclohexane, n-heptane, methylcyclohexane, n-octane and isooctane.
